(19) United States
(12) Patent Application Publication
(10) Pub. No.: US 2011/0125326 A1
(43) Pub. Date: May 26, 2011

(54) USING RAMAN SPECTROSCPPY TO CONTROL CARBONATE/BICARBONATE CONCENTRATIONS

(75) Inventors: Roberto Bosco, Rochester, NH (US); Brian Grandjean, Amesbury, MA (US)

(73) Assignee: POWERSPAN CORP., PORTSMOUTH, NH (US)

(21) Appl. No.: 13/055,642

(22) PCT Filed: Jul. 28, 2009
(86) PCT No.: PCT/US09/51924

§ 371 Date: Jan. 24, 2011

Related U.S. Application Data
(60) Provisional application No. 61/084,524, filed on Jul. 29, 2008.

(51) Int. Cl. C12Q 3/00; G01J 3/44

(52) U.S. Cl. 700/271; 700/266

(57) ABSTRACT

An apparatus and method of controlling carbonate/bicarbonate concentrations and ratio in a chemical process having the steps of flowing a carbonate/bicarbonate solution through a measurement cell (202), exposing the solution to laser light of suitable wavelength and power (204); measuring the intensity of the scattered light using Raman spectroscopy (206); calculating the concentration of carbonate and bicarbonate from the intensity of the scattered light (208); and sending the measurement results to a programmable logic controller (210) to be used to control the ratio of carbonate and bicarbonate in the solution through adjusting process parameters. The method is useful in both carbon dioxide absorption processes and carbonate/bicarbonate regeneration processes.

### BACKGROUND

1. Field of the Invention

The invention relates to detecting chemical composition and controlling chemical processes using Raman spectroscopy.

2. Description of the Related Art

There are a number of useful chemical processes that require carbonate, bicarbonate and total carbonate measurements. One such process is a carbon dioxide scrubbing process in which carbonate and bicarbonate are the main components. Another example is producing carbon dioxide from an alkali carbonate/bicarbonate solution.

The conventional method of determining carbonate, bicarbonate, and total carbonate measurements uses acid/base titration. This is a batch system, which necessarily introduces a finite time lag in concentration measurements. The acid/base titration method is also plagued by interferences from various chemical compounds.

What is needed, therefore, is a continuous, online apparatus and method of detecting and controlling carbonate and bicarbonate concentrations in a chemical process that is not subject to interference from other chemical compounds.

### SUMMARY

The invention is an apparatus and method that satisfies the need for a continuous, online way of detecting and controlling carbonate and bicarbonate concentrations in a chemical process that is not subject to interference from other chemical compounds. One aspect of the invention is a method for controlling a chemical process comprising the steps of flowing a carbonate/bicarbonate mixture through a measurement cell, exposing it with laser light of suitable wavelength and power; measuring the intensity of the scattered light using Raman spectroscopy; calculating the concentration of carbonate and bicarbonate from the intensity of the scattered light, and using the measurement to adjust process control parameters to control the ratio and concentration of bicarbonate and carbonate in the process fluid. These and other features, aspects, and advantages of the present invention will become better understood with regard to the following description, claim, and accompanying drawings.

### DRAWINGS

FIG. 1 is a process flow chart of a process determining bicarbonate/carbonate concentration using Raman spectroscopy according to the present invention.

FIG. 2 is a graph of the Raman spectrum of carbonate and bicarbonate.

FIG. 3 is a process flow chart of online concentration measurement of an alkali carbonate/bicarbonate scrubbing process and absorber control according to the present invention.

FIG. 4 is a process flow chart of online concentration measurement of a carbonate/bicarbonate regeneration process and regenerator control according to the present invention.

### DESCRIPTION

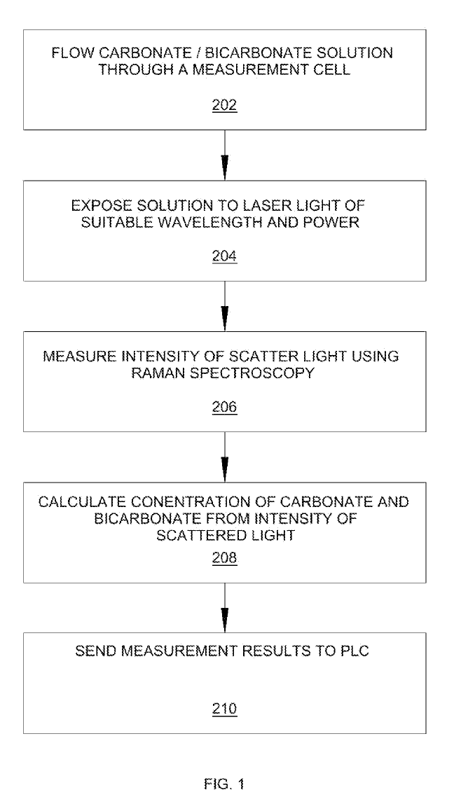

Turning to FIG. 1, one aspect of the invention is a method of controlling carbonate/bicarbonate concentrations in a chemical process having the steps of a) flowing a carbonate/bicarbonate solution through a measurement cell 202, (b) exposing the solution to laser light of suitable wavelength and power 204; c) measuring the intensity of the scattered light using Raman spectroscopy 206; d) calculating the concentration of carbonate and bicarbonate from the intensity of the scattered light 208; and (e) sending the measurement results to a programmable logic controller (“PLC”) 210 to be used to control the ratio and concentration of carbonate and bicarbonate in the solution through adjusting process parameters.

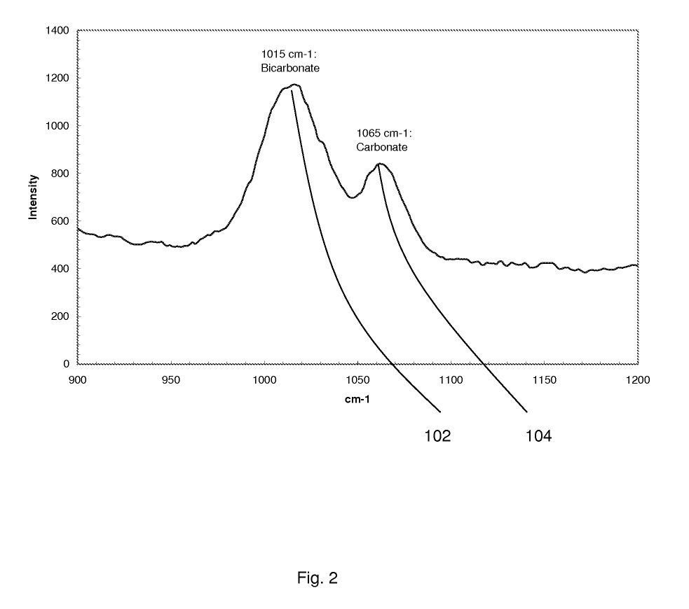

Raman spectroscopy depends upon the inelastic scattering of monochromatic light. The incident light usually comes from a laser in the visible or ultraviolet range. When carbonate and/and bicarbonate are irradiated with the laser light, they shift the frequency of the light. This shift can be measured for both carbonate and bicarbonate and occurs at different frequencies. Turning to FIG. 2, bicarbonate has a peak 102 at approximately 1015 cm⁻¹ and carbonate has a peak 104 at approximately 1065 cm⁻¹.

By measuring the intensity of the scattered light either by peak height or peak area, the concentrations of each component can be determined. These two concentrations can then be used to calculate total carbonate.

The invention involves using Raman spectroscopy to identify and quantify carbonate and bicarbonate real time in a process that relies on the ratio of carbonate, and bicarbonate present as well as the total carbonate concentration. To our knowledge, there are presently no alternatives to performing this online.

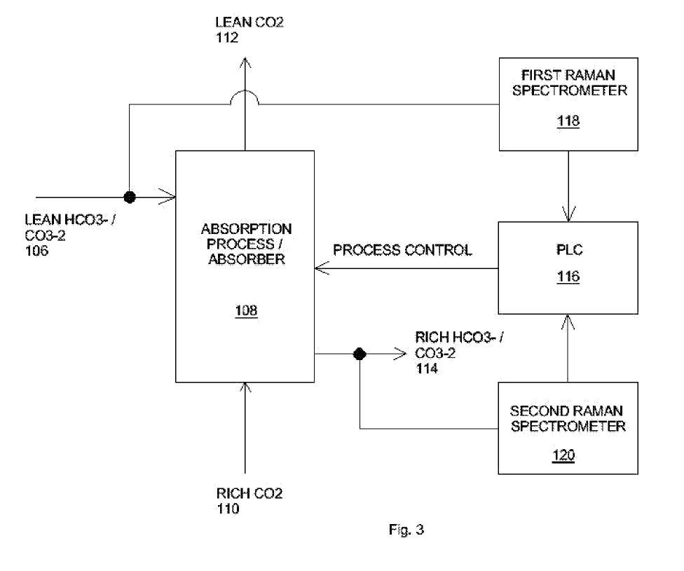

One embodiment is a carbon dioxide scrubbing process where carbonate and bicarbonate are the main components as shown in FIG. 3.

A “carbonate lean” solution 106 is introduced to a CO₂ absorption process/CO₂ absorber 108. A carbonate lean solution contains a ratio of HCO₃⁻/CO₃²⁻ greater than 1. Its composition is measured by a first Raman spectrometer 118. As CO₂ is absorbed into the carbonate solution, the following general reaction will take place:

CO₃⁻²+H₂O+CO₂→2HCO₃⁻ (1)

As this occurs, the total carbonate and carbonate/bicarbonate ratio will change. A “carbonate rich” solution with HCO₃−/CO₃⁻² ratio less than 1 will exit the absorption process 114. Its composition is measured by a second Raman spectrometer 120. The carbonate/bicarbonate solution can be but is not limited to Na₂CO₃/NaHCO₃, (NH₄)₂CO₃/NH₄HCO₃, and K₂CO₃/KHCO₃. The important factor for controlling the ratio of carbonate and bicarbonate in the solution is that the carbonate be soluble in the solution being measured.

This method would be used to control the total carbonate concentration and to control the ratio carbonate concentration to bicarbonate concentration. The concentration values would be sent to a PLC 116 as feedback to the process control loops to the process/absorber 108. These factors are important because if the carbonate to bicarbonate ratio is not controlled it would lead to poor absorption efficiency. If the total carbonate concentration is not controlled, it would lead to “salting out” or precipitation of the carbonate solution fouling mass and heat transfer surfaces. The method provides feedback to the PLC for adjusting parameters such as, but not limited to liquid flow rates, reagent addition rates, and temperatures.

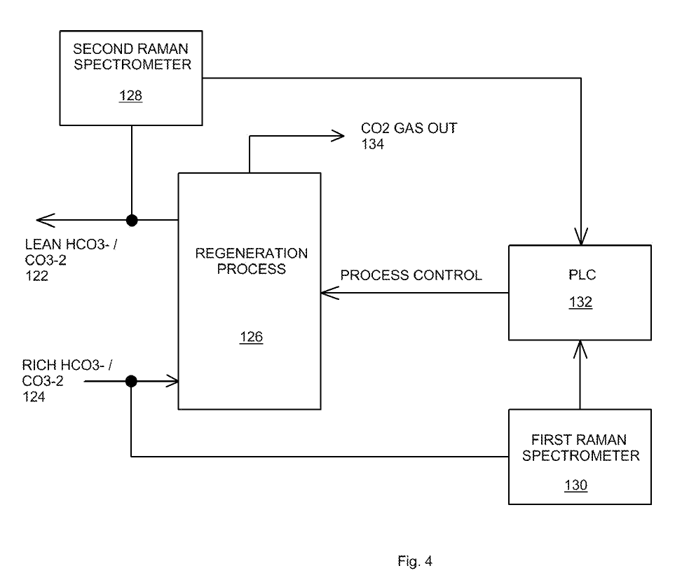

Turning to FIG. 4, another embodiment and process in which this invention can be used is the production of carbon dioxide from alkali carbonate/bicarbonate solutions. This can also be characterized as a carbonate/bicarbonate regeneration process/regenerator 126. To produce CO₂ from alkali carbonate/bicarbonate solutions such as, but not limited to Na₂CO₃/NaHCO₃, (NH₄)₂CO₃/NH₄HCO₃, and K₂CO₃/KHCO₃, the reaction of equation (1) above is reversed. Bicarbonate is converted to carbonate, water and CO₂.

In such a process, feedback will be necessary to determine if the solution has been regenerated 126 to the degree required to by the process. A method of doing this is to measure the carbonate and bicarbonate concentrations along different points of the process.

A rich HCO₃⁻/CO₃⁻² solution 124 is introduced to a regeneration process 126. Its composition is measured by a first Raman spectrometer 130. CO₂ gas 134 is produced as a result of the regeneration process 126. A lean HCO₃⁻/CO₃⁻² solution 122 exits the regeneration process. Its composition is measured by a second Raman spectrometer 128.

Information from the first and second Raman spectrometers 130, 128 would be fed to a PLC 132, which would then control an energy input to the regeneration process 126. The Raman spectrometers would provide real time data input to a PLC or other automated controller that could then be used for either regeneration 126 or absorber 108 control.

One embodiment of a measurement system could include, but not be limited to, the following:
Sample probe;
Data transfer cables from probe to spectrometer;
Spectrometer;
Computer; and
Output to PLC (analog or digital).

Although the preferred embodiments of the present invention have been described herein, the above description is merely illustrative. Further modification of the invention herein disclosed will occur to those skilled in the respective arts and all such modifications are deemed to be within the scope of the invention as defined by the appended claim.

We claim:

1. A method of controlling carbonate/bicarbonate concentrations in a chemical process comprising the steps of:
flowing a carbonate/bicarbonate solution through a measurement cell;
exposing the solution to laser light of suitable wavelength and power;
measuring the intensity of the scattered light using Raman spectroscopy;
calculating the concentration of carbonate and bicarbonate from the intensity of the scattered light; and
sending the measurement results to a programmable logic controller to be used to control the ratio of carbonate to bicarbonate and total carbonate concentration in the solution through adjusting process parameters.

2. A method of controlling bicarbonate/carbonate concentrations in a CO₂ absorption process comprising the steps of:
providing a lean bicarbonate/carbonate solution having a bicarbonate/carbonate ratio of less than 1;
measuring the lean bicarbonate/carbonate composition with a first Raman spectrometer;
absorbing CO₂ with a bicarbonate/carbonate solution, thereby producing a rich bicarbonate/carbonate solution having a bicarbonate/carbonate ratio greater than 1;
measuring the rich bicarbonate/carbonate solution with a second Raman spectrometer; and
sending the lean bicarbonate/carbonate composition measurement and rich bicarbonate/carbonate to a programmable logic controller for controlling the CO₂ absorption process.

3. A method of controlling bicarbonate/carbonate solutions in a bicarbonate/carbonate regeneration process comprising the steps of:
providing a rich bicarbonate/carbonate solution having a bicarbonate/carbonate ratio greater than 1;
measuring the rich bicarbonate/carbonate solution with a first Raman spectrometer;
regenerating the bicarbonate/carbonate solution thereby producing CO₂ and a lean bicarbonate/carbonate solution having a bicarbonate/carbonate ratio of less than 1;
measuring the lean bicarbonate/carbonate solution with a second Raman spectrometer; and
sending the rich bicarbonate/carbonate composition measurement and lean bicarbonate/carbonate to a programmable logic controller for controlling the regeneration process.

4. An apparatus for controlling bicarbonate/carbonate concentrations in a CO₂ absorber comprising:
a CO₂ absorber having as inputs a rich CO₂ stream and lean bicarbonate/carbonate stream, and having as outputs a lean CO₂ stream and rich bicarbonate/carbonate stream;
a first Raman spectrometer in communication with a lean bicarbonate/carbonate input stream for measuring the bicarbonate/carbonate concentration going into the CO₂ absorber;
a second Raman spectrometer in communication with a rich bicarbonate/carbonate out stream output from the CO₂ absorber for measuring the bicarbonate/carbonate concentration leaving the CO₂ absorber; and
a programmable logic controller in communication with the first Raman spectrometer and second Raman spectrometer for receiving bicarbonate/carbonate concentration signals and outputting process control signals to the CO₂ absorber.

5. An apparatus for controlling bicarbonate/carbonate solutions in a bicarbonate/carbonate regeneration process comprising:
a bicarbonate/carbonate regenerator having as inputs a rich bicarbonate/carbonate stream and having as outputs a lean bicarbonate/carbonate stream and CO2;
a first Raman spectrometer in communication with a rich bicarbonate/carbonate input stream for measuring the bicarbonate/carbonate concentration going into the regenerator;
a second Raman spectrometer in communication with a lean bicarbonate/carbonate out stream output from the regenerator for measuring the bicarbonate/carbonate concentration leaving the regenerator; and
a programmable logic controller in communication with the first Raman spectrometer and second Raman spectrometer for receiving bicarbonate/carbonate concentration signals and outputting process control signals to the regenerator.
